(19) United States
(12) Patent Application Publication
(10) Pub. No.: US 2016/0213875 A1
(43) Pub. Date: Jul. 28, 2016

(54) EQUIPMENT FOR TREATING AN OBSTRUCTIVE RESPIRATORY DISORDER

(71) Applicant: PHYSIO-ASSIST, Aix-en-Provence (FR)

(72) Inventors: Jean-Sebastien LANTZ, Aix-en-Provence (FR); Adrien MITHALAL, Montpellier (FR)

(73) Assignee: Physio-Assist, Aix-en Provence (FR)

(21) Appl. No.: 14/916,626

(22) PCT Filed: Sep. 5, 2014
(86) PCT No.: PCT/EP2014/002405

§ 371 Date: Mar. 4, 2016

(30) Foreign Application Priority Data
Sep. 5, 2013 (FR) 13/02063

(51) Int. Cl. A61M 16/06; A61M 16/10

(52) U.S. Cl. CPC A61M 16/0666; A61M 16/107; A61M 2205/11; A61M 2205/6009; A61M 2205/273; A61M 2205/3592

(57) ABSTRACT

Equipment for treating obstructive lung disorders includes:
a respiratory nozzle (2) including at least one air filtering element (21) and at least one transponder (22) on which is recorded a unique identifier and elements for storing information characterising the use of respiratory nozzle, and
a device (1) designed for facilitating expulsion of mucus, and including at least one receiver (11) associated with the transponder and which only allows initiation of the device after detection of the unique identifier, which device includes at least one microcontroller (12) for the management of the transponder, which is designed both for reading information of this transponder and for modifying the information so as to prevent the use of the respiratory nozzle when the latter has already been used once by a patient or to prevent the initiation of the device if the number of uses of respiratory nozzle is greater than a predetermined threshold.

This present international patent application claims priority from French application FR 13/02063 filed Aug. 5, 2013, which is incorporated herein by way of reference.

The present invention relates to the field of treatment of obstructive lung disease.

A patient with obstructive lung disorder, or pulmonary obstructive syndrome (eg., COPD, cystic fibrosis), has a mucus build up. This mucus accumulation is the result of a resistance to this natural mechanism of bronchial drainage and causes a limitation of the flow in the bronchial tree.

In addition to breathing difficulties, this mucus accumulation is likely to result in severe pulmonary complications because stagnation is a breeding ground for bacterial infections.

It is therefore appropriate in the treatment of patients with obstructive respiratory disorder to be particularly vigilant for limiting the risks of super infection.

Some devices have been developed to help these patients in their bronchial clearance (the IPV® (Intrapulmonary Percussive Ventilation) HELP AIR, etc.).

Document US 2009/126731 describes a patient interface assembly comprising a housing unit defining an input and an output port which is connected the patient. This interface is connected via its input port, to a device generating pulses with a frequency ranging from 2 to 25 Hz and in which a pump generates a pressurised air flow. A nebuliser is preferably fluidically connected to the output port and placed for introducing a medicament into the air flow and delivering a medicalised air to the patient.

Document US 2008/066754 describes a high frequency continuous oscillation respiratory device delivering a treatment at the same time during the inhalation and exhalation so as to optimise the cleaning of bronchial secretions. A venturi circuit patient interface is connected with a medicated aerosol for delivering an oscillating high-frequency continuous treatment.

Document US 2005/039749 describes an inhalation-exhalation system for removal of bronchopulmonary secretions with an automatic triggering of the inhalation phase, comprising a conduit for connecting to the airways of a patient, a pressure source with a positive pressure port and a negative pressure port. The system includes a switching device selectively connecting the conduit to the positive pressure port, the negative pressure port to open air, a sensor capable of detecting an inspiration by the patient and a control system for operating the switching device, for connecting lines sequentially to the positive port, the negative port, to the open air and return to the positive port in response to the sensor sensing an inspiration of the patient when the pipe is connected to the open air.

These devices are rarely used now, one of the reasons associated with low usage was the risk of contamination, and cross contamination in particular, related to the use of the same apparatus by several patients.

Patent DE 10 2007 029016 describes a therapeutic respiratory equipment comprising of a mouthpiece which is equipped with a remotely searchable encoding device and permitting the activation of the breathing equipment when it is identified by the latter.

EP patent application 01800705 describes components for a patient ventilation apparatus comprising a remote readable encoding tag. The flow generator controller is programmed to receive data from the identification tag via a tag reader and adjust the flow controller functions to match the component.

The inventors have now developed a solution, adaptable to these devices without altering the performance, to minimise the risk of contamination and cross contamination in particular.

Thus, the present invention is targeted at equipment for obstructive lung disorders including:
a respiratory nozzle (2) comprising at least one air filter holder (21) and at least one transponder (22) on which is recorded a unique identifier, and
a device (1) designed to facilitate the expulsion of mucus, preferably phlegm and comprising at least one receiver (11) which is associated with this transponder (22) and which only allows initiating of the device after detection of said unique identifier,

characterised in that:
said respiratory nozzle (2) further comprises means for storing information characterising the use of respiratory nozzle, and
said device comprises at least one microcontroller (12) for the management of the transponder (22), which is designed both for reading the information of this transponder (22) and for modifying said information so as to prevent the use of the respiratory nozzle (2) when the latter has already been used once by a patient or to prevent the initiation of the device (1) if the number of uses of respiratory nozzle (2) is greater than a predetermined threshold.

Beyond its “classic” definition, the term “obstructive lung disease” can extend here to disorders associated with obstruction of the nasal passages, such as sinusitis and having the same problem. Sinusitis is the obstruction of consecutive sinus inflammation of the mucous membranes of the nose resulting in an impaired drainage of the nasal mucus; in which mucus will then quickly develop bacteria.

“Devices (1) to facilitate the expulsion of mucus”, preferably for facilitating the expectoration of mucus, are well known to those skilled in the art and include the devices described in U.S. Pat. No. 5,862,802 and in the French patent No. 2,733,917.

A “respiratory nozzle” (2) also corresponds to a respiratory mask, to a mouthpiece, to a nosepiece. Now, such a respiratory nozzle will more usually be a respiratory mask or a mouthpiece.

As the air filtering means (21) which may be associated with such a respiratory nozzle (2); it has become widely known and corresponds most often to the filtration membranes. Such filtration membranes are in particular sold by the company MILLIPORE.

In connection with the transponder (22), which is a critical element of the invention, it may take the form of a radio frequency tag or RFID support. Such a tag (holder) comprises in the manner of an induction loop ensuring the supply of a circuit comprising a memory in which is stored a unique identifier. This radio frequency tag or RFID holder is intended to transmit to the device (1) a unique identifier so as to avoid its use by several patients and the transmission of infection between them. For example, such a radio frequency tag operable in the frequency band between 13 and 14 MHz.

At a minimum, the transponder (22) comprising storage means for information characterising the use of the respiratory nozzle (2), information corresponding to one of the following states:
Not used.
or
Used.

The information “Not used” allows the device to commence the facilitation of the expulsion of mucus, while the information “Used” does not allow it.

Now in the case where the equipment of the invention would be in the home of the same patient, for example, one can imagine a transponder (22) allowing for a longer use of respiratory nozzle (2) In such cases, the information stored on the transponder (22) no longer correspond to two states, but the number of uses of the respiratory nozzle (Used 1 time, Used 2 times, . . . , number times used), and the device (1) associated will not get under way if the number of uses is less than a predetermined threshold; then it will enter, at the end of each cycle, additional use on the transponder (22), preferably the RFID holder.

Besides this information, the transponder (22) may also store, for the purpose of transmission to the device (1) parameters related to respiratory nozzle (manufacturing date, expiry date, batch number, etc.) or parameters related to the operation of the device (1) (frequency, power, duration, etc.) In this way, it is possible to associate a transponder (22), and therefore a respiratory nozzle (2) to a given type of patient (COPD, sinusitis, newborn, etc.) so that the device (1), placed in the presence thereof, automatically adjusts the settings for that type of patient and optimises mucus expelled by the patient.

In front, the device (1) is equipped with a power supply that activates the transponder (22) (ex. the radio frequency tag or RFID holder). In the case of a radio frequency tag or RFID holder, this activation will be operated through a magnetic field sensed by the induction loop of the RFID holder. The device of the receiver is then an antenna for receiving the electromagnetic signals transmitted by the radio frequency tag or RFID holder when the latter is powered. The activation of the device (1) is conditioned to detect a transponder identification signal (22). In the absence of such a signal, the device (1) is held in the standby position to prevent, to at least limit, the risk of transmission of infection from one patient to another due to the use of the same respiratory nozzle (2).

Now under the equipment of the invention may include other “consumables” that are also associated with a transponder so as to avoid any risk of transmission of germs to a patient.

We can therefore imagine another air filtering means, directly at the output of the device (1) and constituting a second security; which comprises a further transponder organising the change thereof, for example every month or every 100 uses.

One could also imagine a flexible tube between the device (1) and respiratory nozzle (2), also comprising a third transponder; the latter responder imposing the weekly change or every 25 uses of the hose.

In addition to the system mentioned above, the invention also relates to the respiratory nozzle (2) described previously.

More specifically, the invention relates to a respiratory nozzle (2) comprising at least one air filtering means (21) and at least one transponder (22) on which is recorded a unique identifier and characterised in said transponder (22) comprising further means of storing information characterising the use of respiratory nozzle.

The invention also relates to a method of treating a bronchial obstruction disorder comprising the following steps:
Associate a respiratory nozzle (2) to the device (1)
Approach the respiratory nozzle (2) to the device (1) so that the reading device can read the transponder information holder (22),
Initiate treatment.

Finally, the invention relates to the use of a microcontroller (12) for the management of a transponder (22) of a respiratory nozzle (2) for reading and modification of the information in this transponder (22) in order to prevent the use of respiratory nozzle (2) when the latter has been used a first time or if the number of uses exceeds a predetermined threshold.

Other features, objectives and advantages of the invention will become apparent on reading the detailed description that follows, with reference to the accompanying drawings, given as non-limiting examples and on which:

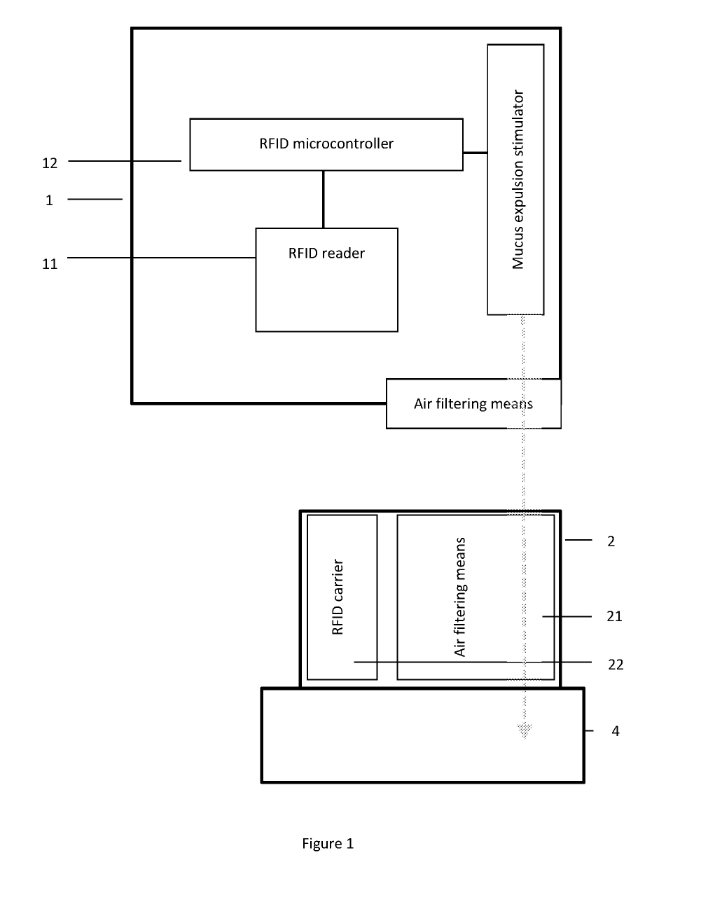

FIG. 1 is a schematic diagram of equipment according to the invention.

The invention is based on the integration of a transponder (22) respiratory nozzle (2) so as to make it accessible to as many devices intended to facilitate mucous expulsion, avoiding or at least limiting the risks of infection and in particular transmission of infection between patients using the same device.

Referring to FIG. 1, the treatment device (1) comprises an RFID reader (11) (in the form of RFID antenna for example) for securing the entire device by allowing the initiation thereof after detecting an RFID holder (22) associated to a respiratory nozzle (2), thus avoiding any risk of infection for the patient (4) to be treated; and secure the treatment by reading the pre-recorded parameters in the holder (22), not allowing another patient to subsequently use the same respiratory nozzle (2). The treatment device (1) also comprises a microcontroller (12) responsible for managing the device (1) and the respiratory nozzle (2).

The respiratory nozzle (2) is in contact with the patient (4). Therefore, it must meet sterility issues, critical with this type of patient (eg cystic fibrosis) and as such that it includes the air filtering means (21). The respiratory nozzle (2) further comprises a RFID holder (22); RFID holder which are widely described in the state of the art. However, the invention also provides that the RFID holder (22) can also have the function to automate the treatment method. The user will be able to possibly select the RFID holder depending on the patient to be treated and the selected holder will deliver to the receiver a signal or information that will drive the device (power, frequency, etc. depending on the pathology and/or patient to be treated). In this context, the RFID holder possesses an identifier that links to a localised table parameter in the device software in which are predefined the duration, frequency and/or the power of stimulation, but also possibly the type of patient who must be treated (for a verification check by the practitioner, for example). When using the device, the user simply selects the suitable respiratory nozzle (which contains the RFID holders) depending on the patient (eg. adult or infant) and type (eg. COPD or sinusitis). Thus, almost no adjustment or settings by the user is required. To further avoid the risk of contamination, a sterilisation process of the respiratory nozzle (2), in particular ethylene oxide, may be proposed.

Referring to FIG. 1 still, the presence of an RFID holder (22) is indicated to the microcontroller 12. The presence of an RFID holder (22) does not give information concerning its status. To know the status of the RFID holder (22), the microcontroller commands a reading of the RFID holder (22). This command allows to know the usage status of RFID holder (22) present in the field reader of the RFID (11). The different status of an RFID holder (22) are:
Used
or
Not Used

It may also include other pieces of information pertaining to the parameter settings of the device.

The microcontroller (12) requests the status of the RFID holder (22) and possibly the mucus stimulation parameters. Depending on the response, the beginning of the stimulation authorised and parameters thereof are adjusted as needed. Simultaneously, the RFID information holder (22) (Used) is updated.

In a context of use, the RFID and respiratory nozzle assembly is also used to perform a so called “double blind” clinical study. In addition to containing the device parameters, the respiratory nozzle contains information specifying if the device should perform a real stimulation of mucus or simply simulate. It is expected that the device for facilitating expulsion of mucus, when the latter actually stimulates the mucus or not, has the same behaviour discernible by the patient. Programming of the mouthpiece is performed during manufacture by software that allows randomly or not the “real” stimulation through the parameters contained in the RFID holder of the respiratory nozzle.

This same software can read the information on the authorisation of stimulating mucus. The practitioner then treats the patient with the help of the respiratory nozzle without knowing whether stimulation is actually delivered by the device. After using the respiratory nozzle, the practitioner gives the respiratory nozzle to a person responsible for the clinical study for evaluation of the results of the stimulation. The person responsible for the study then reads the respiratory nozzle in order to determine if the patient has been stimulated or not.

Thus, with such a process, no one directly taking part can know when the device performs the actual stimulation or not during the clinical trial. This principle allows to gather opinions on the result of the stimulation with complete objectivity.

Of course, all of the numerical values of the description are purely given for information only to guide the implementation of the invention. We can therefore use other numerical values, for example determined by experimentation, without departing from the scope of the invention.

1. Equipment for treating obstructive lung disorders including:
a respiratory nozzle (2) comprising at least one air filtering means (21) and at least one transponder (22) on which is recorded a unique identifier, and
a device (1) designed to facilitate the expulsion of mucus, preferably phlegm, and comprising at least one receiver (11) which is associated with this transponder (22) and which only allows initiating of the device after detection of said unique identifier,
wherein:
said respiratory nozzle (2) further comprises means for storing information characterising the use of respiratory nozzle, and
said device comprises at least one microcontroller (12) for the management of the transponder (22), which is designed both for reading the information of this transponder (22) and for modifying said information so as to prevent the use of the respiratory nozzle (2) when the latter has already been used once by a patient or to prevent the initiation of the device (1) if the number of uses of respiratory nozzle (2) is greater than a predetermined threshold.

2. The equipment according to claim 1, wherein said transponder (22) comprises storage means for information characterising the use of the respiratory nozzle (2), information corresponding to one of the following states:
Not used; or
used.
and where the information not used allows the device to commence (1) while the information “used” does not allow it.

3. The equipment according to claim 1, wherein said transponder (22) comprises means for storing information corresponding to the number of uses of the respiratory nozzle (2), in that the device 1) only starts if the number of uses of the respiratory nozzle (2) is less than a predetermined threshold and in that the device (1) registered at the end of each cycle, an additional use on the transponder (22)

4. The equipment according to claim 1, wherein the transponder (22) is associated with a given type of patient so that the device (1) placed in the presence thereof, automatically adjusts the settings for this type of patient.

5. The equipment according to claim 1, wherein said respiratory nozzle (2) is selected from the group consisting of respiratory masks, mouthpieces, and nasal inserts.

6. The equipment according to claim 1, wherein said transponder (22) is a radio frequency tag or a RFID holder.

7. A respiratory nozzle (2) comprising at least one means of air filter (21) and at least one transponder (22) on which is recorded a unique identifier and wherein said transponder (22) comprises further means of storing information characterising the use of respiratory nozzle.

8. The respiratory nozzle (2) according to claim 7, wherein said transponder (22) comprises means for storing information pertaining to the use of the respiratory nozzle (2), said information corresponding to one of the following states:
Not used; or
Used.

9. The respiratory nozzle (2) according to claim 7, wherein said respiratory nozzle (2) is selected from the group consisting of respiratory masks, mouthpieces, and nasal inserts.

10. The respiratory nozzle (2) according to claim 7, wherein said transponder (22), preferably of said RFID holder or radio frequency holder, includes means preventing a second use of the respiratory nozzle (22) by a device (1) designed to facilitate the expulsion of mucus by a patient.

11. (canceled)

12. The equipment according to claim 2, wherein the transponder (22) is associated with a given type of patient so that the device (1) placed in the presence thereof, automatically adjusts the settings for this type of patient.

13. The equipment according to claim 2, wherein said respiratory nozzle (2) is selected from the group consisting of respiratory masks, mouthpieces, and nasal inserts.

14. The equipment according to claim 2, wherein said transponder (22) is a radio frequency tag or a RFID holder.

15. The equipment according to claim 3, wherein the transponder (22) is associated with a given type of patient so that the device (1) placed in the presence thereof, automatically adjusts the settings for this type of patient.

16. The equipment according to claim 3, wherein said respiratory nozzle (2) is selected from the group consisting of respiratory masks, mouthpieces, and nasal inserts.

17. The equipment according to claim 3, wherein said transponder (22) is a radio frequency tag or a RFID holder.

18. The respiratory nozzle (2) according to claim 8, wherein said respiratory nozzle (2) is selected from the group consisting of respiratory masks, mouthpieces, and nasal inserts.

19. The respiratory nozzle (2) according to claim 8, wherein said transponder (22), preferably of said RFID holder or radio frequency holder, includes means preventing a second use of the respiratory nozzle (22) by a device (1) designed to facilitate the expulsion of mucus by a patient.

20. The respiratory nozzle (2) according to claim 9, wherein said transponder (22), preferably of said RFID holder or radio frequency holder, includes means preventing a second use of the respiratory nozzle (22) by a device (1) designed to facilitate the expulsion of mucus by a patient.
